(19) United States
(12) Patent Application Publication
(10) Pub. No.: US 2024/0009471 A1
(43) Pub. Date: Jan. 11, 2024

(54) SYSTEM TO RECHARGE AN IMPLANTABLE MEDICAL DEVICE

(71) Applicant: VERMON SA, Tours (FR)

(72) Inventors: Bogdan ROSINSKI, Tours (FR); Thien HOANG, Tours (FR); Nicolas FELIX, Tours (FR)

(21) Appl. No.: 18/343,154

(22) Filed: Jun. 28, 2023

(30) Foreign Application Priority Data
Jul. 5, 2022 (FR) 22/06806

(51) Int. Cl. A61N 1/378; A61B 5/00

(52) U.S. Cl. CPC A61N 1/3787; A61B 5/6846; A61B 2560/0219

(57) ABSTRACT

A device to emit ultrasounds to wireless recharge an electronic device, wherein the emitting device includes at least one ultrasonic transducer and an electronic circuit to control the ultrasonic transducer, wherein the control electronic circuit is configured to: apply to the transducer an excitation electric signal in order to induce the emission of an ultrasonic wave towards the electronic device; and read a feedback electric signal generated by the transducer under the action of a part of the ultrasonic wave reflected by the electronic device, wherein the control electronic circuit comprises a feedback loop configured to adjust the frequency of said ultrasonic wave, so that the energy of said reflected part of the ultrasonic wave tends to a minimum value.

### TECHNICAL FIELD

The present disclosure generally relates to devices and systems based on ultrasonic transducers, in particular to a wireless ultrasonic recharge system for an implantable medical device.

### BACKGROUND ART

An implantable medical device is a device designed for implantation in the body of a patient to monitor and/or treat various types of pathologies. Such a device can comprise one or several sensors, for example to measure at least one physiological parameter, and/or one or several actuators, for example to deliver a treatment or to stimulate an organ.

These elements are generally accommodated in a housing made of a biocompatible material, which is implanted in the body of the patient.

An implantable medical device usually comprises a non-rechargeable battery to supply power to its various elements. However, this has limitations in terms of life duration and dimensions.

To reduce the dimensions and/or increase the life duration of an implantable medical device, it has been suggested to use a rechargeable battery powered by a wireless energy transfer system, in particular by an ultrasonic wireless energy transfer system.

It is advisable to reduce, at least partially, some aspects of known solutions of wireless ultrasonic recharging of implantable medical devices.

### SUMMARY OF INVENTION

To do so, an embodiment provides a device to emit ultrasounds to wireless recharge an electronic device, wherein the emitting device comprises at least one ultrasonic transducer and an electronic circuit to control said at least one ultrasonic transducer, wherein the control electronic circuit is configured to:
apply to the ultrasonic transducer an excitation electric signal in order to induce the emission, by the ultrasonic transducer, of an ultrasonic wave towards the electronic device;
read a feedback electric signal generated by the ultrasonic transducer under the action of a part of the ultrasonic wave reflected by the electronic device,
   wherein the control electronic circuit comprises a feedback loop configured to adjust the frequency of said ultrasonic wave, so that the energy of said reflected part of the ultrasonic wave tends to a minimum value.

According to an embodiment, the control electronic circuit is configured to apply to said ultrasonic transducer an excitation electric signal with frequency modulation, whose frequency continuously varies between a frequency fₛₜₐᵣₜ and a frequency fₛₜₒₚ higher than fₛₜₐᵣₜ.

According to an embodiment, the feedback loop comprises a treatment circuit configured to detect the frequency f_{NZR} where the energy of the reflected part of the ultrasonic wave is minimum.

According to an embodiment, the treatment circuit is additionally configured to adjust the values of the frequencies fₛₜₐᵣₜ and fₛₜₒₚ so that they approach the frequency f_{NZR}, thus reducing the energy of the reflected part of the ultrasonic wave.

According to an embodiment, the control circuit comprises a detection circuit of the envelope of said feedback electric signal generated by the ultrasonic transducer.

According to an embodiment, the control circuit comprises a detection circuit of edges of the envelope of said feedback electric signal generated by the ultrasonic transducer.

According to an embodiment, the control electronic circuit is configured to apply to said ultrasonic transducer an impulse excitation electric signal.

According to an embodiment, the control electronic circuit is configured to apply a continuous excitation electric signal to the ultrasonic transducer.

According to an embodiment, the ultrasonic transducer comprises an emission transducer and a reception transducer.

According to an embodiment, the ultrasonic transducer comprises a single transducer for the emission of the ultrasonic wave and for the reception of the reflected part of the ultrasonic wave.

### BRIEF DESCRIPTION OF DRAWINGS

The foregoing features and advantages, as well as others, will be described in detail in the following description of specific embodiments given by way of illustration and not limitation with reference to the accompanying drawings, in which:

FIG. 1 is a diagram that schematically shows the energy conversion yield of an ultrasonic transducer as a function of the acoustic frequency of the received wave and the electric charge of the transducer;

FIG. 2 illustrates schematically, as a block diagram, an example of a device to emit ultrasounds to wirelessly recharge an implantable medical device; and

FIG. 3 is a diagram that illustrates an example of operation of the device of FIG. 2.

### DESCRIPTION OF EMBODIMENTS

Like features have been designated by like references in the various figures. In particular, the structural and/or functional features that are common among the various embodiments may have the same references and may dispose identical structural, dimensional and material properties.

For the sake of clarity, only the operations and elements that are useful for an understanding of the embodiments described herein have been illustrated and described in detail. In particular, the manufacture of the ultrasonic transducers and of the control electronic circuits of the described devices has not been described, since the described embodiments are compatible with the usual manufactures of these elements or since a person skilled in the art will be able to manufacture these elements starting from the specifications of the present disclosure.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

Unless specified otherwise, the expressions “around”, “approximately”, “substantially” and “in the order of” signify within 10%, and preferably within 5%.

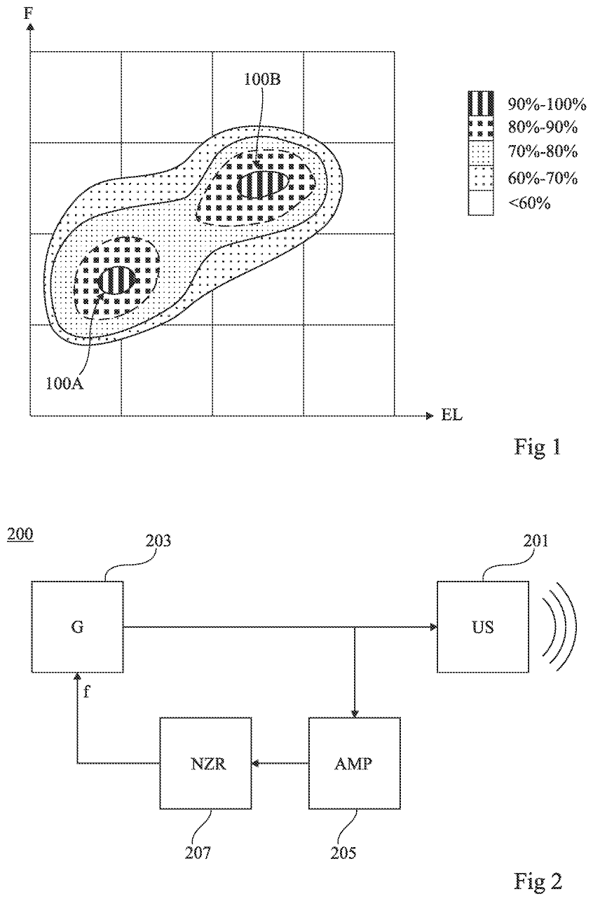

FIG. 1 is a diagram that schematically shows the energy conversion yield of an ultrasonic transducer (by range in percentage of acoustic energy converted into electric energy, illustrated as different texturings), as a function of the acoustic frequency F of the wave received by the transducer, in ordinate, and of the electric charge EL as detected by the transducer, in abscissa.

The transducer is, for example, integrated in an implantable medical device (not illustrated) to position into the body of a patient. The implantable medical device comprises, for example, an electronic circuit connected to the terminals of the transducer and configured to convert electric signals generated at the terminals of the transducer under the action of a received acoustic wave into electric signals to power or recharge a battery of the implantable medical device.

The acoustic wave is, for example, emitted by an emission device located out of the body of the patient. This allows the implementation of a wireless ultrasonic recharge of the battery of the implantable medical device.

As shown in FIG. 1, the conversion yield of the acoustic energy from the emission device into electric energy to power the implantable device has peaks (maximum values) for given values of the acoustic frequency of the received wave and of the electric charge connected to the transducer. In particular, in this illustrated example, the conversion yield has two peaks 100A and 100B that correspond respectively to a resonance mode and an antiresonance mode of the reception transducer.

These optimal values of acoustic frequency and electric charge may vary among devices or in time, notably because of deviations of manufacturing and variations of the acoustic impedance of the ambient. Furthermore, the electric charge detected by the transducer may vary in time, notably as a function of the power consumption of the implantable medical device.

Thus, when implementing the wireless ultrasonic recharging of an implantable medical device, it is advisable that the emission acoustic frequency of the external device is dynamically adjustable, in order to maximize the yield of the energy transmission chain.

To do so, it is possible to provide a communication link, for example a wireless communication link, for example relying on radio-frequency signals (RF) or ultrasonic signals. This way, the implantable device is able to transmit the external device an information feedback about the received amount of electric energy. Thus, the emission device is able to adjust the ultrasonic emission frequency based on this information, in order to maximize the conversion yield. However, this process has a drawback resulting from more cost and more electric consumption in relation to the wireless communication circuit.

The part of the acoustic energy emitted by the emission device and not converted into electric power by the transducer integrated to the implantable medical device is, at least partially, reflected by implantable medical device towards the emission source.

According to another embodiment, the emission device is configured to measure the part of the ultrasonic wave reflected by the implantable medical device and adjust the emission frequency so that the energy of said reflected part of the ultrasonic wave tends to a minimum value. This makes it possible to have the yield tend to a maximum value for the complete chain of transmission of electric power towards the implantable medical device.

FIG. 2 schematically illustrates, as blocks, an example of a device 200 to emit ultrasounds to wireless recharge an implantable medical device according to an embodiment.

The device 200 comprises at least one ultrasonic transducer 201 (US) configured to emit ultrasonic waves towards an implantable medical device to recharge. The transducer 201 is additionally configured to receive ultrasonic waves from the implantable medical device. In particular, following the emission of a recharging ultrasonic wave towards the implantable medical device, the ultrasonic transducer is suitable to receive a part of said reflected wave by the implantable medical device.

Preferably, the transducer 201 comprises two ultrasonic transducers respectively dedicated to the emission of a recharging ultrasonic wave towards the implantable medical device and to the reception of a part of said reflected wave by the implantable medical device. As a variant, the emission and the reception are manufactured by a same ultrasonic transducer.

The transducer 201, for example, is a piezoelectric transducer. More generally, the modes for carrying out the invention can be adapted to suit any type of ultrasonic transducer, for example capacitive ultrasound transducers with membrane (CMUT), piezoelectric ultrasound transducers with membrane (PMUT), etc.

In addition, the device 200 comprises a circuit 203 (G) to generate excitation electrical signals applied to the transducer 201 to initiate the emission of the recharging ultrasonic wave by the transducer 201. In particular, the circuit 203 is configured to adapt the frequency of the excitation electric signals generated according to a setpoint f in order to adjust the acoustic frequency of the recharging ultrasonic wave emitted by the transducer 201.

The device 200 also comprises a feedback loop configured to read a feedback electric signal generated by the transducer 201 under the effect of the part of the recharging ultrasonic wave reflected by the implantable medical device, and to consequently adapt the setpoint frequency of the circuit 203, and consequently the emission frequency of the transducer 201, in order to tend toward a minimum value of the reflected part of the recharging ultrasonic wave.

In the illustrated example, the feedback loop comprises an amplifier 205 (AMP) configured to amplify the feedback electric signal generated by the transducer 201 under the action of a part of the recharging ultrasonic wave reflected by the implantable electronic device.

In this example, the feedback loop also comprises a treatment circuit 207 (NZR) receiving the amplified feedback signal from the amplifier 205 and configured to generate a signal representative of the energy of the reflected portion of the recharging ultrasonic wave. The circuit 207 is configured to adjust the frequency setpoint f provided to the circuit 203 in order to minimize the energy of the reflected part of the recharging ultrasonic wave.

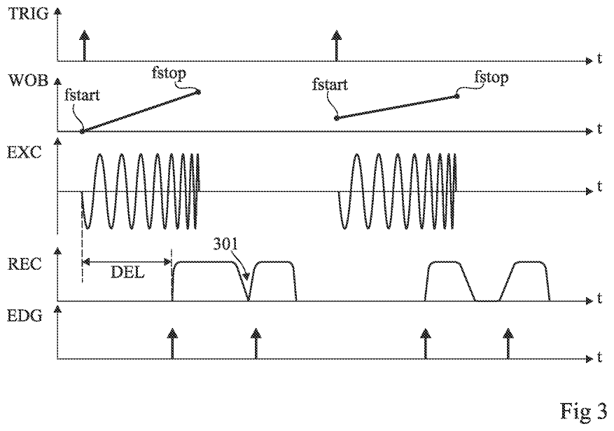

FIG. 3 is a diagram that illustrates an example of operation of the device 200 of FIG. 2.

In this example, the ultrasonic acoustic wave emitted by the emission device 200 has the shape of burst waves. As an example, the frequency of the repetition of pulses is stationary, and the duty cycle of the pulses (or the duration of the pulses) is stationary.

FIG. 3 more specifically illustrates the evolution in time t (in abscissa):
of a pulse triggering signal TRIG,
of a signal WOB representative of the setpoint frequency applied to the circuit 203 that generates the excitation signal of the transducer 201,
of the excitation signal EXC generated by the circuit 203,
of a signal REC representative of the envelope of the part of the recharging ultrasonic wave reflected by the implantable medical device, and
of a signal EDG representative of the rising edge of the envelope signal REC.

In this example, the excitation signal emitted by the generator 203, hence the ultrasonic signal emitted by the transducer 201 with frequency modulation, which means that the emission frequency varies, for example continuously, for example linearly, between a frequency f start and a frequency fₛₜₒₚ, for example higher than fₛₜₐᵣₜ, between the start and the end of each impulsion.

After each impulsion, a part of the ultrasonic wave emitted by the transducer 201 is reflected towards the transducer. Thus, a feedback signal is generated at the terminals of the transducer 201. This feedback signal is amplified by the amplifier circuit 205. The signal REC corresponds to the envelope of the amplified feedback signal. The treatment circuit 207 comprises, for example, an analogical circuit to detect envelopes, configured to generate the signal REC from the amplified feedback signal provide by the amplification circuit 205.

As illustrated in FIG. 3, the feedback signal shows a delay DEL relatively to the excitation signal EXC, which corresponds to the duration required for the round-trip propagation of the wave between the transducer 201 and the implantable medical device.

The signal REC is representative of the energy of the part of the recharging ultrasonic wave reflected by the implantable medical device. As illustrated in FIG. 3, for each pulse, the signal REC shows a dip or local minimum that matches an absorption peak of the ultrasonic signal by the implantable device at a frequency between fₛₜₒₚ and fₛₜₐᵣₜ. This minimum value corresponds to a resonance or antiresonance mode of the implantable medical device (mode 100A or 100B of FIG. 1), where the energy conversion yield is optimal.

For each impulsion of the ultrasonic signal that the implantable medical device reflects, the signal EDG comprises a first rising edge that corresponds to the start of the impulsion, and a second rising edge that corresponds to the end of the dip of the signal REC. The treatment circuit 207 comprises, for example, an edge detection circuit configured to generate the signal EDG from the signal REC.

As an example, the treatment circuit 207 is configured to analyze the signal EDG and deduce the frequency f_{NZR} where the reflection of the ultrasonic wave by the implantable medical device is minimum.

The treatment circuit 207 is, for example, configured to adjust the start frequency fₛₜₐᵣₜ and end frequency fₛₜₒₚ of the modulated pulse signal, in order to tend them toward the frequency f_{NZR} and so reduce the ultrasonic energy reflected by the implantable medical device, hence increase the yield of the electric energy transmission chain transmission to the implantable medical device.

As a variant, the excitation signal emitted by the generator 203, hence the ultrasonic signal emitted by the transducer 201, has a fixed frequency f during all the impulsion. In this case, the treatment circuit 207 can be configured to have the frequency f vary between two impulsions, so that the acoustic energy reflected by the implantable medical device tend to a minimum value, for example based on a principle of PLL (Phase-Locked Loop).

In another variant, the ultrasonic wave emitted by the transducer 201 can be a continuous wave, and not an impulse wave. In this case, as in the example above in relationship with FIG. 3, the ultrasonic wave can be a frequency modulated wave, which means that its frequency continuously varies, for example linearly, between a frequency f start and a frequency fₛₜₒₚ. The cycle of variation between the frequencies fₛₜₐᵣₜ and fₛₜₒₚ, also called modulation cycle, can have a constant duration. Similarly as what was described about FIG. 3, at each cycle, the treatment circuit 207 detects the frequency where the acoustic energy reflected by the implantable medical device is minimum and adjusts accordingly the frequencies fₛₜₐᵣₜ and fₛₜₒₚ for the following cycle.

As a variant, the continuous ultrasonic wave emitted by the transducer 201 has a fixed adjustable frequency (not modulated). In this case, the treatment circuit 207 can be configured to have the frequency f vary in time, so that the acoustic energy reflected by the implantable medical device tend to a minimum value.

Although it is described, in relationship with FIG. 3, a solution of analogical treatment of the feedback signal generated by the transducer 201, the described embodiments do not restrict to this specific case.

As a variant (not illustrated), the feedback loop of the device 200 can comprise an A/D converter, for example at the output of the amplifier 205, configured to digitalize the feedback signal representative of the wave reflected by the implantable medical device. Then, a digital treatment circuit comprising, for example, a microcontroller, can implement the detection of the frequency of minimal reflection. This allows the implementation of more complex feedback algorithms.

Various embodiments and variants have been described. Those skilled in the art will understand that certain features of these embodiments can be combined and other variants will readily occur to those skilled in the art. In particular, although only an example of application to ultrasonic wireless recharging of an implantable medical device is described above, the described embodiments do not restrict to this specific case. More generally, the described embodiments can apply to any ultrasonic wireless recharging system of an electronic device, either medical or not, and implantable or not.

1. A device to emit ultrasounds to wireless recharge an electronic device, wherein the emitting device comprises at least one ultrasonic transducer and an electronic circuit to control said ultrasonic transducer, wherein the control electronic circuit is configured to:
apply to said at least one ultrasonic transducer an excitation electric signal (EXC) in order to induce the emission, by said at least one ultrasonic transducer, of an ultrasonic wave towards the electronic device;
read a feedback electric signal generated by said at least one ultrasonic transducer under the action of a part of said ultrasonic wave reflected by the electronic device;
generate a signal representative of the energy of the reflected portion of the ultrasonic wave,
wherein the control electronic circuit comprises a feedback loop configured to adjust the frequency of said ultrasonic wave, so that the energy of said reflected part of the ultrasonic wave tends to a minimum value.

2. The device according to claim 1, wherein the control electronic circuit is configured to apply to said at least one ultrasonic transducer an excitation electric signal (EXC) with frequency modulation, whose frequency continuously varies between a frequency fₛₜₐᵣₜ and a frequency fₛₜₒₚ higher than fₛₜₐᵣₜ.

3. The device according to claim 2, wherein the feedback loop comprises a treatment circuit configured to detect the frequency f_{NZR} where the energy of said reflected part of the ultrasonic wave can be minimized.

4. The device according to claim 3, wherein the treatment circuit is additionally configured to adjust the values of the frequencies f start and fₛₜₒₚ so that they approach the frequency f_{NZR}, thus reducing the energy of the reflected part of the ultrasonic wave.

5. The device according to claim 1, wherein the control circuit comprises a detection circuit of the envelope of said feedback electric signal generated by said at least one ultrasonic transducer.

6. The device according to claim 5, wherein the control circuit comprises a detection circuit of edges of said envelope of said feedback electric signal generated by said at least one ultrasonic transducer.

7. The device according to claim 1, wherein the control electronic circuit is configured to apply to said at least one ultrasonic transducer an impulse excitation electric signal (EXC).

8. The device according to claim 1, wherein the control electronic circuit is configured to apply to said at least one ultrasonic transducer a continuous excitation electric signal (EXC).

9. The device according to claim 1, wherein said at least one ultrasonic transducer comprises an emission transducer and a reception transducer.

10. The device according to claim 1, wherein said at least one ultrasonic transducer comprises a single transducer for the emission of said ultrasonic wave and for the reception of said reflected part of said ultrasonic wave.
